(19) United States
(12) Patent Application Publication
(10) Pub. No.: US 2024/0324679 A1
(43) Pub. Date: Oct. 3, 2024

(54) METHODS AND DEVICES FOR CONTROLLING THE TEMPERATURE OF A DRUG FOIL SUBSTRATE TO GENERATE AN AEROSOL

(71) Applicant: ALEXZA PHARMACEUTICALS, INC., Fremont, CA (US)

(72) Inventors: David Koji HASEGAWA, Fremont, CA (US); Andreas KNAPIC, Dornbirn (AT); Mingzhu LEI, Fremont, CA (US); Ken MOCHEL, Fremont, CA (US)

(73) Assignee: ALEXZA PHARMACEUTICALS, INC., Fremont, CA (US)

(21) Appl. No.: 18/742,331

(22) Filed: Jun. 13, 2024

Related U.S. Application Data
(63) Continuation of application No. PCT/US2022/052909, filed on Dec. 14, 2022.
(60) Provisional application No. 63/289,606, filed on Dec. 14, 2021.

(51) Int. Cl. A24F 40/46; A24F 40/42; A24F 40/51

(52) U.S. Cl. CPC A24F 40/46; A24F 40/42; A24F 40/51

(57) ABSTRACT

A method of controlling the temperature of a drug foil substrate characterized by stopping the heating while measuring its temperature deciding the amount of time that the drug foil substrate is heated in the following period is disclosed.

### FIELD

The present methods and devices belong to the field of control temperature for aerosol generation of a drug.

### BACKGROUND

In CN1548934 A a sensor and a circuit to detect the temperature of a heating element are described. Measuring the heating is stopped and the heating element is allowed to stabilize the temperature for a suitable period of time.

In WO03037412A2 a method of controlling the temperature profile of a resistive element for vaporising a medicament is disclosed. Said method includes a pulse heating with a first high duty cycle for the ramp up and a reduced duty cycle to maintain the temperature. The timing of temperature determination is not specified. The device produces a cool central region and a hot region around the centre.

In WO07076688 A1 a method of controlling the temperature of a heating element where the electrical resistance changes monotonically with temperature and where the temperature is sensed after the heating has stopped is described.

In WO14040988 A2 a method of maintaining the temperature of an aerosol generating device providing pulses of electrical current (duty cycle) is described. The method includes determining the temperature of the heating element by determining its resistance and the duty cycle is reduced when the temperature is closer to the target temperature by adjusting the electrical current, e.g., by limiting the voltage applied. The aerosol-forming substrate is based on tobacco.

In US2021219386 AA and US2021145074 an apparatus including two switches; a first switch to interrupt the flow of current to heat a resistive heating element that causes vaporization of a vaporizable material and a second switch to add a reference resistor in series with the resistive heating element is described. The apparatus allows to determine the resistance of the resistive heating element and controlling the amount of power supplied to the resistive heating element based on the determined resistance by adjusting the flow of current.

In WO2015192084A1 an electronic vaporizer including a heating element to heat a fluid to produce a vapour as described. Said vaporizer includes a power control unit configured to regulate the electrical power supplied to the heating element. The regulation is based at least in part in the temperature of the heating element and the temperature setting in order to avoid that the heating element exceeds its temperature setting. The temperature of the heating element may be measured using its resistance based on the known temperature coefficient of resistance characteristics associated with the heating element, the temperature may be determined as often as once every 100 ms. In a different embodiment, the vaporizer includes a machine-readable medium with the temperature coefficient resistance stored in it. The main objective of the vaporizer is to avoid overheating the vapour and maintain the temperature around the boiling point of the fluid. An electronic vaporizer including a heating element to heat a fluid to produce a vapour is described. Said vaporizer includes a power control unit configured to regulate the electrical power supplied to the heating element. The regulation is based at least in part in the temperature of the heating element and the temperature setting in order to avoid that the heating element exceeds its temperature setting. The temperature of the heating element may be measured using its resistance based on the known temperature coefficient of resistance characteristics associated with the heating element, the temperature may be determined as often as once every 100 ms. In a different embodiment, the vaporizer includes a machine-readable medium with the temperature coefficient resistance stored in it. The main objective of the vaporizer is to avoid overheating the vapour and maintain the temperature around the boiling point of the fluid.

In WO2019152873 a disposable cartridge with a drug coated on an electrically heated drug foil substrate is described. The disposable cartridge can be connected to a handheld controller which includes the electronics and electrical components, including the electricity source. The drug foil substrate is heated with a ramp-up to a target temperature and then a heating rate is applied. The heating rate is selected between plateau heating, tampered cooling, and progressive heating. However, the document does not describe how to accurately control the temperature during the ramp-up and heating rate resulting in a high dispersion of the emitted dosc.

The heating of a drug foil substrate to generate an aerosol may be affected by the voltage of the battery which may be different depending on its charging level and may vary, as well, during any given heating cycle if high currents are applied.

The current output of the battery, which as well affects the heating of a drug foil substrate to generate an aerosol, may change depending on its charge level and during any given heating cycle when high currents are applied.

Further, depending on the ambient temperature the heat dissipation from the drug foil substrate may vary from inhalation to inhalation and even during a given inhalation.

Not only that, but the process when the drug of the foil substrate evaporates absorbs energy (known as enthalpy of vaporization), and that extra energy needs to be added in a precise moment, which cannot be foretold, and not in another moment.

Further, on one side, if the heating duration is too short the drug foil substrate may not rest enough time at the temperature required for the evaporation of the drug and the emitted dose will be lower than that required for the therapy, if it is too long the drug foil substrate may rest longer time at the temperature required for the evaporation of the drug and the emitted dose will be higher than the required for the therapy and possibly with more impurities.

Additionally, on another side, if the drug foil substrate is overheated, the aerosol produced may have increased impurities and the emitted dose may be higher than that required for the therapy, on the other side if it is underheated the emitted dose may be lower than that required for the therapy.

In condensation aerosol delivery systems, the aerosol quality of some drugs is very sensitive to the temperature used to vaporize the drug (see e.g., Comparative Example 1 and FIG. 1G). The temperature heating profile (temperature ramp rate, peak temperature, and peak temperature dwell time) all influence the amount of drug that is vaporized and the purity of the aerosol. If the peak temperature is too high and/or if the dwell time is too long, aerosol purity can be negatively impacted. If the peak temperature is too low and/or if the dwell time is too short, the amount of drug vaporized may be too low.

In the prior art, electric heating is mainly used in the field of e-cigarettes to heat tobacco, herbal derivatives, or liquid formulations. The temperature control in that field is important in order to provide a suitable particle size and aerosol dosing, but in the field of drug administration, it is much more important to accurately control those parameters in order to administer a precise and reproducible dose of the drug in order to achieve its therapeutic effect without any undesired side effect. Further, the purity of the administered drug is as well of capital importance in the field of drug administration.

Thus, there is a need for a method and device capable of accurately controlling the heating profile of a drug foil substrate so that it is at the desired temperature at each moment of the heating profile.

### SUMMARY

A first aspect discloses a method of electrically heating a drug foil substrate (205), coated with a solid drug film (207), placed in a disposable cartridge (200) following a first temperature vs. time profile (TP1) to generate a condensation aerosol; the disposable cartridge (200) is suitable to be connected to a handheld controller (100);
TP1 including:
   a heating function with an average slope HSTP1 that ranges between 1 and 5° C./ms;
   a target temperature tTTP1 that ranges between 20° and 550° C., and/or
   a heating time HtTP1 that ranges between 40 and 550 ms;
the method including:
   a) a regulation phase including:
      applying an electric current ITP1 to the drug foil substrate (205) during t_heat1 and not applying any current during t_wait1,
      not applying any current during t_wait1 and applying an electric current ITP1 to the drug foil substrate (205) during t_heat1, or
      the previous t_wait1 and t_heat1 are distributed in alternate sections;
   b) determining the temperature of the drug foil substrate (205) (T_meas) during t_meas1 using a temperature sensor;
   c) if T_meas≤ T_exp then
      t_heat1=1-100% of (t_period1−t_meas1) and t_wait1=t_period1−t_meas1−t_heat1,
      if not
      t_heat1=0-100% of (t_period1−t_meas1) and t_wait1=t_period1−t_meas1−t_heat1,
where T_exp is based on TP1; and
   d) repeating steps a) to d) to match TP1 until tTTP1 is reached and/or HtTP1 has elapsed;
where:
   the control period time t_period1 ranges between 0.5 and 5 ms,
   the measurement time t_meas1 ranges between 1-40% of t_period1,
   T_exp is the temperature expected at any given time point based on TP1,
   t_period1=t_heat1+t_wait1+t_meas1,
   t_period1 is the length of time of each cycle of measuring temperature and optionally heating the drug foil substrate (205),
   t_heat1 is the time the drug foil substrate (205) is heated within each t_period1,
   t_wait1 is the time the drug foil substrate (205) is not heated within each t_period1,
   t_meas1 is the time the temperature of the drug foil substrate (205) is measured within each t_period1, and
   the electric current ITP1 is between 30 and 400 A.

A second aspect is a disposable cartridge (200) configured to perform the method of the first aspect and suitable to be connected to the handheld controller (100) of a third aspect including:
an air inlet (220) at one end of the airway (203);
an air outlet (202), configured as a mouthpiece, at another end of the airway (203);
a drug foil substrate (205) having an impermeable surface, with or without perforations, placed within the airway (203);
a drug foil substrate support (204);
a solid drug film (207) coated on the drug foil substrate (205); and
electrical (302) and/or data connections (301) between the disposable cartridge (200), and the handheld controller (100) of the third aspect.

A third aspect is a handheld controller (100) configured to perform the method of the first aspect and suitable to be connected to the disposable cartridge (200) of the second aspect including:
at least one battery (101)
at least one microcontroller (102); and
electrical (302) and/or data connections (301) between the at least one battery (101), the at least one microcontroller (102), and the disposable cartridge (200) of the second aspect.

A fourth aspect is a medicament including a drug selected from: loxapine, alprazolam, estazolam, fentanyl, zaleplon, almorexant, apomorphine, pergolide, ropinirole, pramipexol, granisetron, ondansetron, palonosetron, nicotine, nicotine meta-salicylate, rotigotine, or its pharmaceutically acceptable salts for use in the method of the first aspect, the handheld controller (100) of the third aspect or the disposable cartridge (200) of the second aspect.

The fifth aspect is a drug deposited on a drug foil substrate (205) of the method of the first aspect, the handheld controller (100) of the third aspect or the disposable cartridge (200) of the second aspect for use in a condition or episode, where the drug is:
A. loxapine or its pharmaceutically acceptable salts, the condition or episode is agitation, including:
   i. rapidly controlled mild to moderate agitation in adults with schizophrenia or bipolar disorder, or
   ii. acute agitation associated with schizophrenia or bipolar disorder in adults;
B. alprazolam, estazolam or its pharmaceutically acceptable salts, the condition or episode is epilepsy, where epilepsy includes seizures;
C. fentanyl or its pharmaceutically acceptable salts, the condition or episode is breakthrough pain;
D. zaleplon, almorexant or its pharmaceutically acceptable salts, the condition or episode is a sleep disorder including:
   i. middle of the night awakening, or
   ii. middle of the night insomnia;
E. apomorphine, pergolide, ropinirole, pramipexol, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
   i. nausea,
   ii. vomiting or
   iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexol, or rotigotine, the condition or episode is restless legs syndrome.

### Definitions

Within the present document, the following terms are used with the following meanings.
i. Disposable cartridge” is understood to be a single dose or multidose cartridge including a drug coated on a drug foil substrate, an air inlet that may or may not be connected to a handheld controller, an airway (203), an air outlet configured as a mouthpiece and connectors to electrically connect it to the handheld controller (100). The disposable cartridge is attached to a handheld controller in order to be operated.
ii. “Handheld controller” is a reusable device including at least one battery, connectors, electrical parts, and electronics suitable to heat the drug foil substrate once a disposable cartridge has been attached to it.
iii. “Electrical connections” are means to transfer electrical power from a battery to a drug foil substrate, a microcontroller, or a memory.
iv. “Data connections” are means to transfer data between a microcontroller and any sensor, detector, or memory, where the sensor or detector and memory are in the disposable cartridge and/or the handheld controller.
v. “Battery” means a device for storing energy in chemical compounds capable of generating an electrical current. It can be a rechargeable battery, such as nickel-metal hydride, lithium-polymer battery, or the like (see WO2019152873 for further details on rechargeable batteries); or a single use battery, such as a dry cell battery, alkaline batteries, silver cells batteries, zinc-air batteries, lithium batteries, nickel oxyhydroxide batteries and the like. When the heating to vaporize a drug is chemical heating the battery is needed to start the chemical reaction and/or control the electronics of the device.
vi. “Drug foil substrate” is an impermeable surface where a drug is deposited to be heated and aerosolized. If the heating is electric, further information on electric heating can be found in WO2019152873, incorporated herein by reference. If the heating is chemical, it may be referred to as chemical heating pack, further information on chemical heating packs can be found in WO2004104492, incorporated herein by reference. The drug foil substrate defines a “drug foil substrate plane” which sometimes is preferably substantially parallel to the aerosol axis (223), i.e., defining an angle of −10 to 10°. The drug foil substrate may be planar or curved.
vii. “Solid drug film” is a layer which includes a pure drug, two or more drugs, or one or more drugs in combination with additional components. Additional components can include, for example, pharmaceutically acceptable excipients, carriers, and the like.
viii. “Average slope” is the slope of a line that intersects room temperature at time 0 and the target temperature of a given profile at the heating time.
ix. “Step-function” is a line-function with very high slope (towards infinite).

### BRIEF DESCRIPTION OF FIGURES

FIG. 1A illustrates a Device 1: SS, electrical bench-Top Screening Device.

FIG. 1B illustrates a Device 2: handheld housing 1.

FIG. 1C illustrates a Device 3: handheld housing 2.

FIG. 1D illustrates a heat Source 1: Testing electric heating.

FIG. 1E illustrates a heat Source 2: Chemical heat package.

FIG. 1F illustrates a heat Source 3: Electric heating.

FIG. 1G is a plot of the purity of the emitted aerosol versus the temperature of the drug foil substrate of Comparative Example 1.

FIG. 2A illustrates a handheld medical device including a disposable cartridge (200) and a handheld controller (100) where the air inlet (220) of the disposable cartridge is connected to the air inlet extension (103) of the handheld controller (100).

FIG. 2B illustrates a handheld medical device including a disposable cartridge (200) and a handheld controller (100) where the air inlet (220) of the disposable cartridge is directly open to the atmosphere.

FIG. 3A illustrates the time allocation within t_period1.

FIG. 3B illustrates the drug foil substrate (205) resistance (R205) measurement circuit.

FIG. 4 illustrates a flow chart to follow profile TP1.

FIG. 5A illustrates a t_period1 of an embodiment in which the temperature is measured based on the resistance of the drug foil substrate (205). The drawing is not to scale.

FIG. 5B illustrates a t_period1 of an embodiment in which the temperature is measured based on the resistance of the drug foil substrate (205). The drawing is not to scale.

FIG. 5C illustrates a t_period1 of an embodiment in which the temperature is measured based on the resistance of the drug foil substrate (205). The drawing is not to scale.

FIG. 5D illustrates a t_period1 of an embodiment in which the temperature is measured based on the resistance of the drug foil substrate (205). The drawing is not to scale.

FIG. 6A illustrates an ideal plot of the temperature measured (T_meas, continuous line) and the expected temperature (T_exp, dashed line) using an embodiment where when T_meas is higher than T_exp the following t_period1 heats ⅓ of the t_period1-t_meas1 time. For the sake of simplicity, t_meas1 has not been included in this figure.

FIG. 6B illustrates an ideal plot of T_meas (continuous line) and T_exp (dashed line) using an embodiment where each t_period1 is either heating or not heating. For the sake of simplicity, t_meas1 has not been included in this figure.

FIG. 7 illustrates a flow chart to follow profile TP2.

FIG. 8A illustrates a t_period2 of an embodiment in which the temperature is measured based on the resistance of the drug foil substrate (205). The drawing is not to scale.

FIG. 8B illustrates a t_period2 of an embodiment in which the temperature is measured based on the resistance of the drug foil substrate (205). The drawing is not to scale.

FIG. 8C illustrates a t_period2 of an embodiment in which the temperature is measured based on the resistance of the drug foil substrate (205). The drawing is not to scale.

FIG. 8D illustrates a t_period2 of an embodiment in which the temperature is measured based on the resistance of the drug foil substrate (205). The drawing is not to scale.

FIG. 9 illustrates a flowchart followed by a device which embodies profiles TP1 and TP2.

FIG. 10 illustrates a plot of the temperature of the drug foil substrate (continuous line) and T_exp (dashed line) of a drug foil substrate using an embodiment as described in Example 1. The temperature of the drug foil substrate is measured using a thermal camera (Thermacam A655sc, FLIR).

FIG. 11 illustrates a plot of the temperature of 9 drug foil substrates (continuous lines) and T_exp (dashed line) of a drug foil substrate using an embodiment as described in Example 2. The temperature of the drug foil substrate is measured

### DETAILED DESCRIPTION

FIG. 2A illustrates a handheld medical device including a disposable cartridge (200) attached to a handheld controller (100). The disposable cartridge (200) includes an air inlet (220), an airway (203), and an air outlet configured as a mouthpiece (202). The handheld controller (100) includes the drug foil substrate heating circuit including a battery (101), a microcontroller (102); and an air inlet controller extension (103). The air inlet controller extension (103) and the air inlet (220) are fluidly and tightly connected. The handheld controller (100) and the disposable cartridge (200) are connected via an electric (302) and, optionally, a data connections (301) to share electricity and, optionally, data.

FIG. 2B is similar to FIG. 2A but without the air inlet controller extension (103) in the handheld controller (100). The air inlet (220) in the disposable cartridge (200), which may contain a baffle, is directly open to the atmosphere.

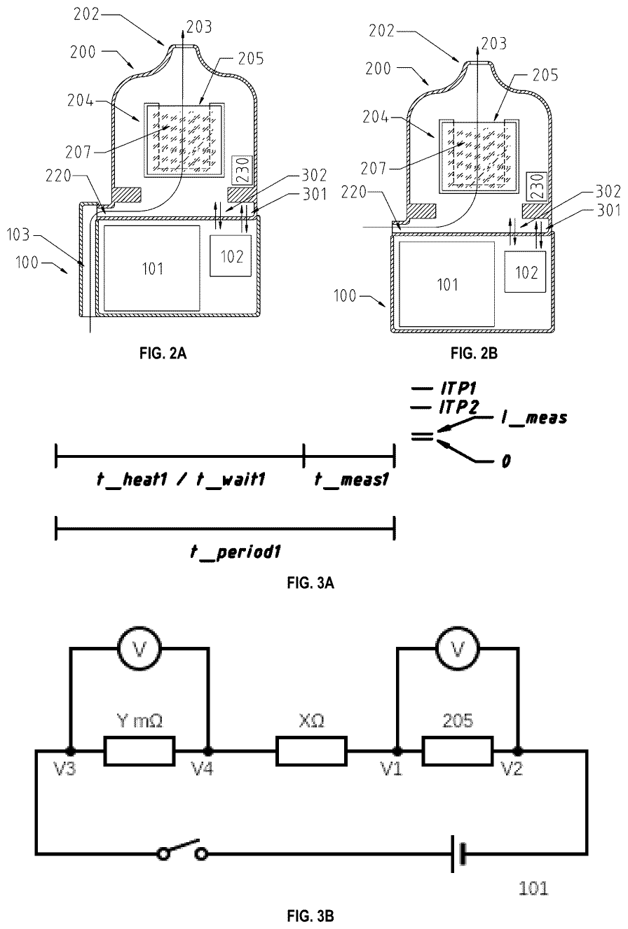

The first temperature profile (TP1) includes several t_period1, each of which, as illustrated in FIG. 3A, are divided in three different sections, one for heating (t_heat1), one for not applying any current (t_wait1) and one for measurement (t_meas). The t_heat1 to t_wait1 ratio will depend on the circumstances and can even be all t_wait1 or all t_heat1. TP2 is divided analogously.

The temperature can be calculated using measurement of electrical resistance across the drug foil substrate (205), optical measurement, direct contact measurement with a thermocouple and/or any other method known.

The resistance of the drug foil substrate is measured e.g., using a circuit as the one illustrated in FIG. 3B using the following well-known Ohm's law formula:

V=I·R

The measurement circuit (see FIG. 3B) includes a X Ω resistor to reduce the measurement current to I_meas A, dependent on the voltage (Vbattery) of the battery (101):

$$I\_meas = \frac{{V}_{battery}}{X}$$

The measurement of the voltage drop (V3−V4) due to the known resistor Y Ω (see FIG. 3B), whose resistance is selected to be close to the resistance of the drug foil substrate (205), is used to measure I_meas:

$$I\_meas = \frac{{V}_{4} - {V}_{3}}{Y}$$

I_meas is used, along with the voltage drop around the drug foil substrate, V2−V1 (see FIG. 3B), to calculate the resistance (R205) of the drug foil substrate (205):

$$I\_meas = \frac{{V}_{2} - {V}_{1}}{{R}_{205}}$$

On summary, the resistance (R205) of the drug foil substrate (205) can be calculated as (see FIG. 3B):

$${R}_{205} = Y \cdot \frac{{V}_{2} - {V}_{1}}{{V}_{4} - {V}_{3}}$$

The drug foil substrate has a resistance vs. temperature relationship, i.e., at each temperature it has a different resistance. This relationship can be determined for every drug foil substrate (or a number of them) during manufacturing of the disposable cartridge by heating the drug foil substrate (205) to a set of predetermined temperatures and measuring the corresponding resistance, and the relationship stored in the disposable cartridge.

The temperature can be measured as well using an optical measurement, i.e., by detecting the amount of infrared energy emitted by the drug foil substrate. Its emission properties can be determined for every drug foil substrate (or a number of them) during manufacturing of the disposable cartridge and the relationship stored in the disposable cartridge.

The temperature can be measured as well using direct contact measurement with a thermocouple calibrated for the desired temperature range.

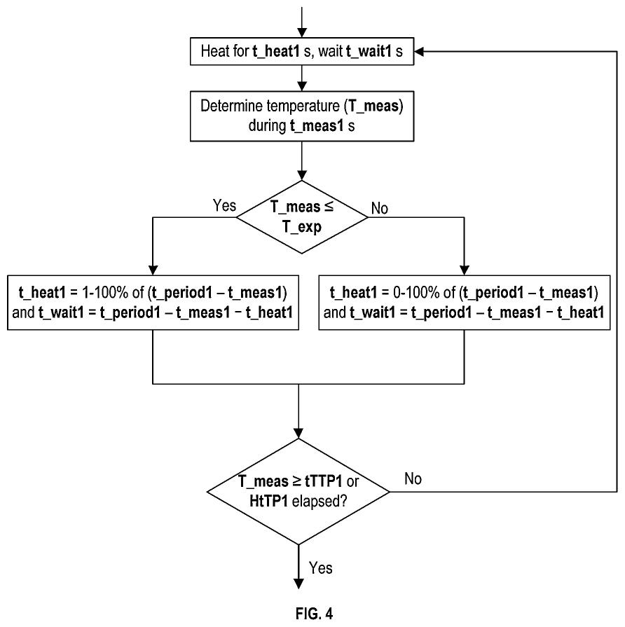

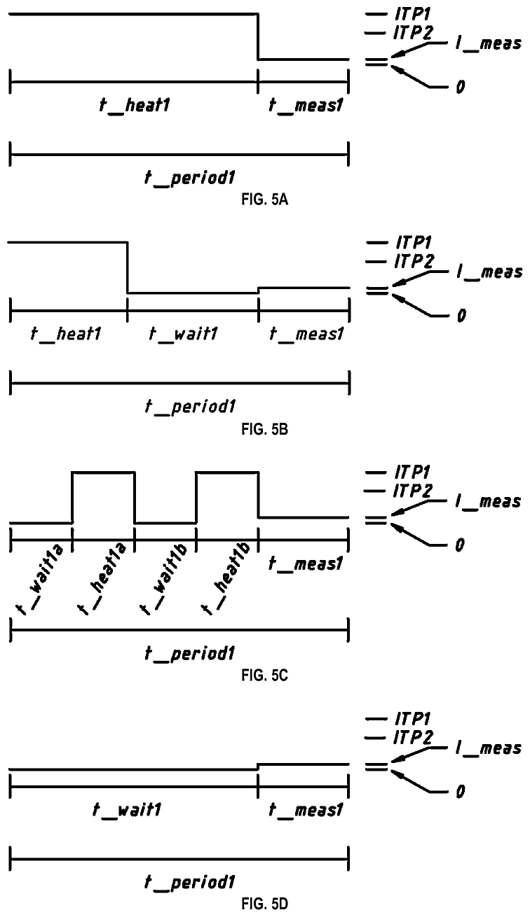

TP1 is illustrated in the flowchart in FIG. 4. The method starts by heating during a time t_heat1 and not applying current during a time t_wait1 (FIG. 5B) or not applying current during a t_wait1 time and then heating during a time t_heat1 (FIG. 5C), although any combination is possible or even distributing t_heat1 and t_wait1 in different sections (see FIG. 5C).

In TP1 an embodiment is that one of t_heat1 and t_wait1 is 0 and the other one is t_period1-t_meas1 (see FIGS. 5A and 5D), although any combination is possible, e.g., part of t_period1 is t_heat1 and the other part is t_wait1 (see FIG. 5B), or even distributing t_heat1 and t_wait1 in different sections (see FIG. 5C). The value of t_period1 may vary during TP1.

The next step in FIG. 4 is determining the temperature T_meas (as described above) during t_meas1. T_meas is compared with T_exp (the expected temperature) for this moment according to the temperature time relationship based on TP1, and the next t_heat1 and t_wait1 are set. if T_meas≤ T_exp then if T_meas≤ T_exp then t_heat1=1-100% of (t_period1−t_meas1) and t_wait1=t_period1−t_meas1−t_heat1; if not t_heat1=0-100% of (t_period1−t_meas1) and t_wait1=t_period1−t_meas1−t_heat1.

FIG. 5A shows a _period1 where t_wait1=0 and t_heat1=t_period1−t_meas.

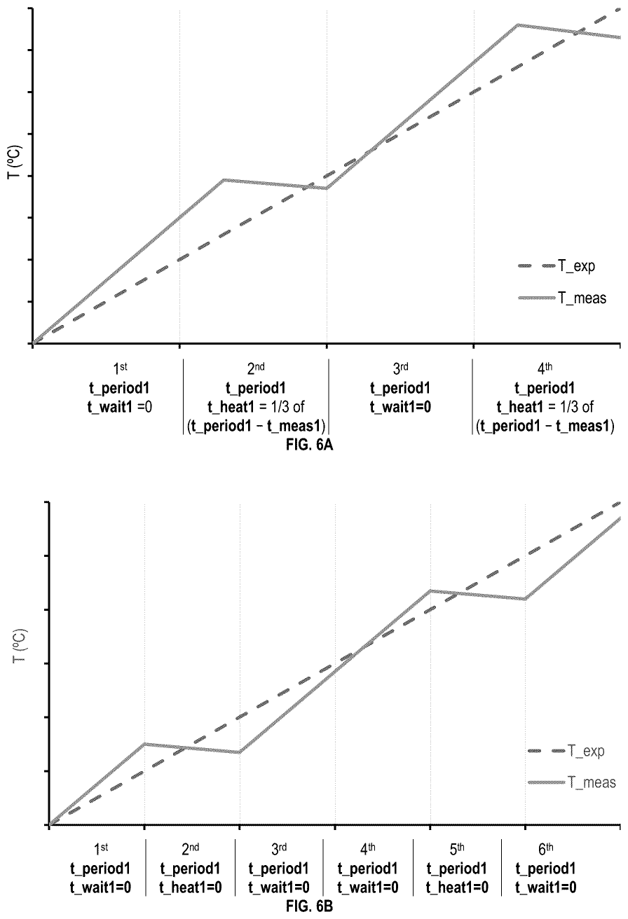

The selection of ratio between t_heat1 and t_wait1 for the second case depends on the performance of the drug (sensibility to temperature, enthalpy of vaporization, and the like) to be vaporized. If it desired that the temperature is not much higher than T_exp, then if T_meas≥T_exp then t_heat1=0 (such as in FIG. 5D) following a profile similar to the representative profile depicted in FIG. 6B. In the first t_period1 of FIG. 6B t_wait1=0, which means that t_heat1=(t_period1−t_meas1), the same is true for the third, fourth and sixth t_period1 of FIG. 6B. In the second t_period1 of FIG. 6B t_heat1=0, which means that t_wait1=(t_period1−t_meas1), the same is true for the fifth t_period1 of FIG. 6B.

Sometimes it is preferred that the temperature is not much lower than T_exp, then if T_meas≥T_exp then t_heat1 can be selected based on the temperature slope of the previous t_period1 so that at the end of the following t_period1 the T_exp (dash line in FIG. 6A) at that time is reached following a profile similar to the representative profiled illustrated in FIG. 6A. In the first t_period1 of FIG. 6A t_wait1=0, which means that t_heat1=(t_period1−t_meas1), the same is true for the third t_period1 of FIG. 6A. In the second t_period1 of FIG. 6A t_heat1=⅓ of (t_period1−t_meas1) which means that t_wait1=⅔ of (t_period1−t_meas1), the same is true for the fourth t_period1 of FIG. 6A.

Any other ratio between t_heat1 and t_wait1 can be selected depending on the drug to be vaporized. Furthermore, the ratio between t_heat1 and t_wait1 may vary during TP1, for instance it may be desirable to reduce t_heat1 and increase t_wait when approaching tTTP1 in order to avoid surpassing said tTTP1. FIGS. 5B and 5C illustrate two possible options to allocate t_heat1 and t_wait1. In FIG. 5B t_heat1 is at the beginning of t_period1 and in FIG. 5C t_heat1 and t_wait1 have been distributed in two sections each.

The process is repeated until the desired temperature is reached (T_meas≥ tTTP1) or the time for TP1 (HtTP1) has elapsed.

If after the tTTP1 has been reached or HtTP1 has elapsed, it is necessary to control the temperature for longer time or follow subsequent temperature profile, then TP2 is followed.

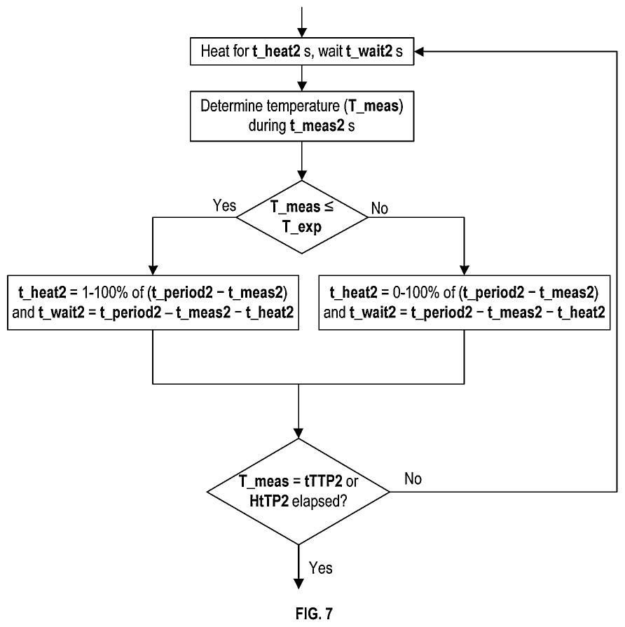

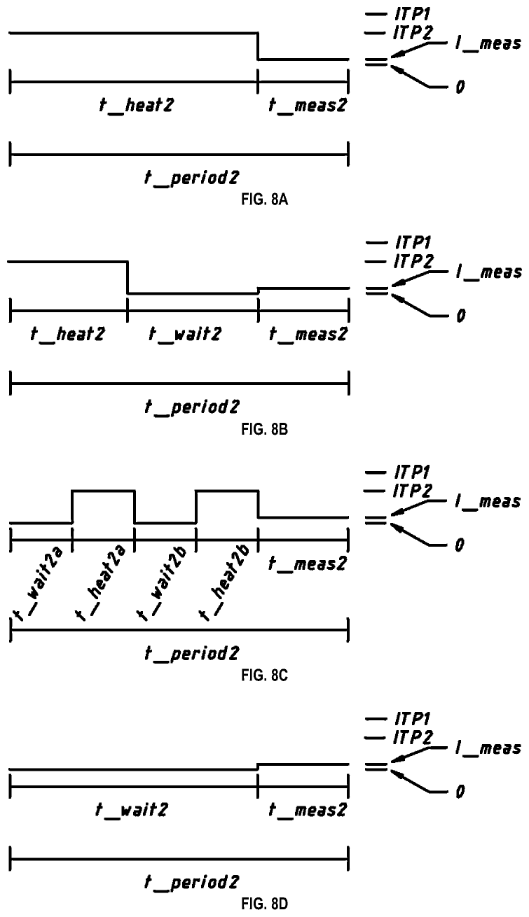

The flowchart for method following TP2 is illustrated in the flow chart in FIG. 7. The method starts by heating during a time t_heat2 and not applying a current during a time t_wait2 (FIG. 8B) or not applying current during a t_wait2 time and then heating during a time t_heat2 (FIG. 8C), although any combination is possible or even distributing t_heat2 and t_wait2 in different sections (see FIG. 8C).

In TP2, an embodiment is that both of t_heat2 and t_wait2 are different from 0 and the other one is t_period2−t_meas2 (see FIG. 8D), although combination is possible, e.g., part of t_pediod2 is t_heat2 and the other part is t_wait2 (see FIG. 8B), or even distributing of t_heat2 and t_wait2 in different sections (see FIG. 8C). The value of t_period2 may vary during TP2.

The next step in TP2 (FIG. 7) is determining the temperature T_meas during t_meas2 similarly as described for TP1.

Then T_meas is compared with the expected temperature for this moment according to the temperature time relationship based on TP2, T_exp, and the next t_heat2 and t_wait2 are set. if T_meas≤T_exp then t_wait2=098% and t_heat1=1−98% of t_period2 (such as in FIG. 8A-8C), if not t_wait2=1−99% and t_heat1=0-98% of t_period1 (such as in FIG. 8A-8D).

The selection of the ratio between t_heat2 and t_wait2 for the second case depends on the performance of the drug (sensibility to temperature, enthalpy of vaporization, and the like) to be vaporized. If it desired that the temperature is not much higher than T_exp, then if T_meas≥T_exp then t_heat2=0 (such as in FIG. 8D) following a profile analogous to the representative profile depicted in FIG. 6B for t_period1. Sometimes it is preferred that the temperature is not much lower than T_exp, then if T_meas≥T_exp then t_heat1 can be selected based on the temperature slope of the previous t_period2 so that at the end of the following t_period2 the T_exp at that time is reached following a profile analogous to the representative profiled illustrated in FIG. 6A for t_period1. Any other ratio between t_heat2 and t_wait2 can be selected depending on the drug to be vaporized and the ration may vary during TP2. FIGS. 8B and 8C illustrate two possible options to allocate t_heat2 and t_wait2. In FIG. 8B t_heat2 is at the beginning of t_period2 and in FIG. 8C t_heat2 and t_wait2 have been distributed in two sections each.

The process is repeated until the desired temperature is reached (T_meas≥ tTTP2) or the time for TP2 (HtTP2) has elapsed.

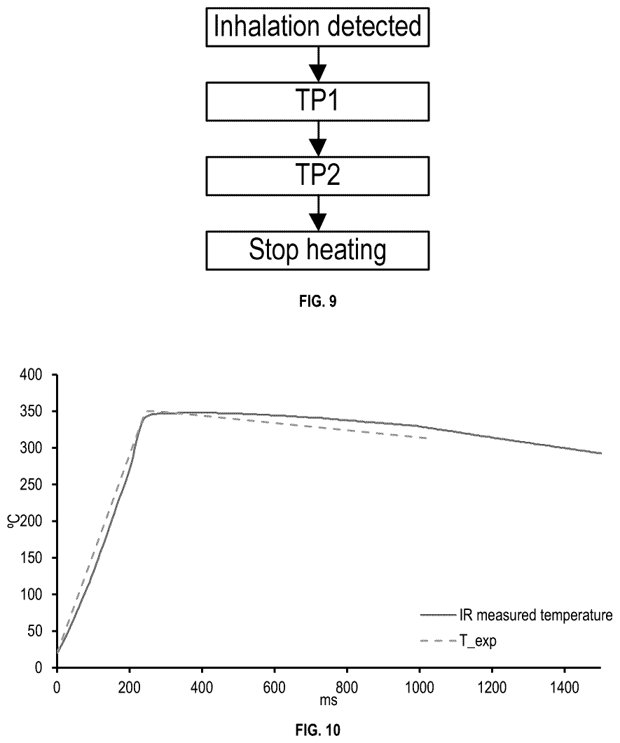

A process of aerosol generation is illustrated in FIG. 9, once the inhalation is detected profile TP1 is followed and when finished the, optionally, profile TP2 is followed and when TP2 is completed the heating is stopped.

Embodiment 1. A method of electrically heating a drug foil substrate (205), coated with a solid drug film (207), placed in a disposable cartridge (200) following a first temperature vs. time profile (TP1) to generate a condensation aerosol; the disposable cartridge (200) is suitable to be connected to a handheld controller (100);
TP1 including:
   a heating function with an average slope HSTP1 that ranges between 1 and 5° C./ms; and
   a target temperature tTTP1 that ranges between 20° and 550° C., and/or
   a heating time HtTP1 that ranges between 40 and 550 ms;
the method including:
   a. a regulation phase including:
      applying an electric current ITP1 to the drug foil substrate (205) during t_heat1 and not applying any current during t_wait1,
      not applying any current during t_wait1 and applying an electric current ITP1 to the drug foil substrate (205) during t_heat1, or
      the previous t_wait and t_heat1 are distributed in alternate sections;
   b. determining the temperature of the drug foil substrate (205) (T_meas) during t_meas1 using a temperature sensor;
   c. if T_meas≤ T_exp then t_heat1=1-100% of (t_period1−t_meas1) and t_wait1=t_period1−t_meas1−
   t_heat1,
   if not
   t_heat1=0-100% of (t_period1−t_meas1) and t_wait1=t_period1−t_meas1−
   t_heat1,
where T_exp is based on TP1; and
   d. repeating steps a) to d) to match TP1 until tTTP1 is reached and/or HtTP1 has elapsed;
where:
   the control period time t_period1 ranges between 0.5 and 5 ms,
   the measurement time t_meas1 ranges between 1-40% of t_period1, T_exp is the temperature expected at any given time point based on TP1,
   t_period1=t_heat1+t_wait1+t_meas1,
   t_period1 is the length of time of each cycle of measuring temperature and optionally heating the drug foil substrate (205),
   t_heat1 is the time the drug foil substrate (205) is heated within each t_period1,
   t_wait1 is the time the drug foil substrate (205) is not heated within each t_period1,
   t_meas1 is the time the temperature of the drug foil substrate (205) is measured within each t_period1, and
   the electric current ITP1 is between 30 and 400 A.

Embodiment 2. The method of the previous Embodiment which further includes a second temperature vs. time profile (TP2) immediately after the end of TP1;
TP2 including:
   a heating function with an average slope HSTP2 that ranges between −0.15 and 0.15° C./ms; and
   a target temperature tTTP2 that ranges between 20° and 400° C., and/or
   a heating time HtTP2 that ranges between 0.5 and 2000 ms;
the method including:
   A. a regulation phase including:
      applying an electric current ITP2 to the drug foil substrate (205) during t_heat2 and not applying any current during t_wait2,
      not applying any current during t_wait2 and applying an electric current ITP2 to the drug foil substrate (205) during t_heat2, or
      the previous t_wait2 and t_heat2 are distributed in alternate sections;
   B. determining the temperature drug foil substrate (T_meas) during t_meas2 using a temperature sensor;
   C. if T_meas≤ T_exp then
   t_heat2=1-100% of (t_period2−t_meas2) and t_wait2=t_period2−t_meas2-t_heat2,
   if not
   t_heat2=0-100% of (t_period2−t_meas2) and t_wait2=t_period2−t_meas2−t_heat2,
where T_exp is based on TP2; and
   D. repeating steps A) to D) to match TP2 until tTTP2 is reached and/or HtTP2 has elapsed;
where
   the control period time t_period2 ranges between 0.5 and 5 ms,
   the measurement time t_meas2 ranges between 1-40% of t_period2,
   t_period2=t_heat2+t_wait2+t_meas2,
   t_period2 is the length of time of each cycle of measuring temperature and optionally heating the drug foil substrate (205),
   t_heat2 is the time the drug foil substrate (205) is heated within each t_period2,
   t_wait2 is the time the drug foil substrate (205) is not heated within each t_period2,
   t_meas2 is the time the temperature of the drug foil substrate (205) is measured within each t_period2, and
   the electric current ITP2 ranges between 10 and 50 A.

Embodiment 3. The method according to any of the previous Embodiments, where the heating function in TP1 is a line, a power function, polynomial function, step-function, or a logarithmic function; or in another embodiment the function is a line.

Embodiment 4. The method according to any of the previous Embodiments, where HSTP1 ranges between 1.25 and 4.5° C./ms, in another embodiment between 1.5 and 4.0° C./ms, in another embodiment between 1.75 and 3.5° C./ms, or in another embodiment between 1.75 and 3.0° C./ms.

Embodiment 5. The method according to any of the previous Embodiments, where tTTP1 ranges between 200 and 400° C., in another embodiment between 225 and 390° C., in another embodiment between 25° and 380° C., in another embodiment between 275 and 370° C., or in another embodiment between 30° and 360° C.

Embodiment 6. The method according to any of the previous Embodiments, where HtTP1 ranges between 40 and 400 ms, in another embodiment between, 50 and 350 ms, in another embodiment between 75 and 300 ms, in another embodiment between 90 and 250 ms in another embodiment between 100 and 200 ms, or in another embodiment between 125 and 180 ms.

Embodiment 7. The method according to any of the previous Embodiments, where t_period1 ranges between 0.55 and 4.0 ms, in another embodiment between 0.6 and 3.5 ms, in another embodiment between 0.7 and 3.0 ms, in another embodiment between 0.75 and 2.5 ms, in another embodiment between 0.8 and 2.0 ms, or in another embodiment between 0.9 and 1.75 ms.

Embodiment 8. The method according to any of the previous Embodiments, where t_meas1 ranges between 5 and 35% of t_period1, in another embodiment between 10 and 30% of t_period1, in another embodiment between 15 and 25% of t_period1, or in another embodiment between 15 and 22% of t_period1.

Embodiment 9. The method according to any of the previous Embodiments, where ITP1 ranges between 30 and 300 A, in another embodiment between 50 and 290 A, in another embodiment between 75 and 280 A, in another embodiment between 100 and 280 A, in another embodiment between 125 and 260 A, in another embodiment between 150 and 250 A, or in another embodiment between 175 and 240 A.

Embodiment 10. The method according to any of the previous embodiments, where when T_meas is lower than tTTP1, then c) is if T_meas≤ T_exp then t_heat1=90100% of (t_period1−t_meas1) and t_wait1=t_period1−t_meas1−t_heat1, if not t_heat1=0% of (t_period1−t_meas1) and t_wait1=t_period1−t_meas1−t_heat1.

Embodiment 11. The method according to any of the previous Embodiments, where when T_meas is higher than (TTP1−50° C. and lower than (TTP1−20° C., then c) is if T_meas≤T_exp then t_heat1=7090% of (t_period1−t_meas1) and t_wait1=t_period1−t_meas1−t_heat1, if not t_heat1=0% of (t_period1−t_meas1) and t_wait1=t_period1−t_meas1−t_heat1.

Embodiment 12. The method according to any of the previous Embodiments, where when T_meas is higher than (TTP1−20° C., then c) is if T_meas≤ T_exp then t_heat1=40-60% of (t_period1−t_meas1) and t_wait1=t_period1−t_meas1−t_heat1, if not t_heat1=0% of (t_period1−t_meas1) and t_wait1=t_period1−t_meas1-t_heat1.

Embodiment 13. The method according to any of the previous Embodiments, when T_meas is lower than tTTP1−50° C., step c) is
if T_meas≤ T_exp then t_heat1=2-100% of (t_period1−t_meas1) and t_wait1=t_period1t_meas1t_heat1, if not t_heat1=0-80% of (t_period1-t_meas1) and t_wait1=t_period1-t_meas1-t_heat1;
in another embodiment if T_meas≤T_exp then t_heat1=4-100% of (t_period1−t_meas1) and t_wait1=t_period1−t_meas1−t_heat1, if not t_heat1=0-60% of (t_period1−t_meas1) and t_wait1=t_period1−t_meas1−t_heat1;
in another embodiment if T_meas≤T_exp then t_heat1=6-100% of (t_period1−t_meas1) and t_wait1=t_period1−t_meas1−t_heat1, if not t_heat1=0-40% of (t_period1−t_meas1) and t_wait1=t_period1−t_meas1−t_heat1;
in another embodiment if T_meas≤ T_exp then t_heat1=8-100% of (t_period1−t_meas1) and t_wait1=t_period1−t_meas1−t_heat1, if not t_heat1=0-20% of (t_period1−t_meas1) and t_wait1=t_period1−t_meas1−t_heat1;
in another embodiment if T_meas≤T_exp then t_heat1=100% of (t_period1−t_meas1) and t_wait1=t_period1−t_meas1−t_heat1, if not t_heat1=0-20% of (t_period1−t_meas1) and t_wait1=t_period1−t_meas1-t_heat1;
in another embodiment if T_meas≤T_exp then t_heat1=100% of (t_period1−t_meas1) and t_wait1=t_period1−t_meas1−t_heat1, if not t_heat1=0% of (t_period1−t_meas1) and t_wait1=t_period1−t_meas1−t_heat1,
in another embodiment if T_meas≤ T_exp then t_heat1=70-90% of (t_period1−t_meas1) and t_wait1=t_period1−t_meas1−t_heat1, if not t_heat1=0% of (t_period1−t_meas1) and t_wait1=t_period1−t_meas1−t_heat1; or
in another embodiment if T_meas≤ T_exp then t_heat1=40-60% of (t_period1−t_meas1) and t_wait1=t_period1−t_meas1−t_heat1, if not t_heat1=0% of (t_period1−t_meas1) and t_wait1=t_period1−t_meas1−t_heat1.

Embodiment 14. The method according to any of Embodiment 2 to Embodiment 13, where the heating function in TP2 is a line, a power function, polynomial function, step-function, or a logarithmic function; in another embodiment the function is a line.

Embodiment 15. The method according to any of Embodiment 2 to Embodiment 14, where HSTP2 ranges between −0.13 and 0.13° C./ms, in another embodiment between −0.1 and 0.1° C./ms, in another embodiment between −0.08 and 0.08° C./ms, or in another embodiment between −0.05 and 0.08° C./ms.

Embodiment 16. The method according to any of Embodiment 2 to Embodiment 15, where tTTP2 ranges between 20° and 400° C., in another embodiment between 225 and 390° C., in another embodiment between 25° and 380° C., in another embodiment between 275 and 370° C., or in another embodiment between 30° and 360° C.

Embodiment 17. The method according to any of Embodiment 2 to Embodiment 16, where HtTP2 ranges between 0.5 and 1500 ms, in another embodiment between 0.5 and 1000 ms, in another embodiment between 1.5 and 500 ms, in another embodiment between 2.5 and 250 ms, in another embodiment between 3.0 and 125 ms, in another embodiment between 4.5 and 75 ms, in another embodiment between 5.0 and 50 ms, or in another embodiment between 5.5 and 25 ms.

Embodiment 18. The method according to any of Embodiment 2 to Embodiment 17, where t_period2 ranges between 0.55 and 4.0 ms, in another embodiment between 0.6 and 3.5 ms, in another embodiment between 0.7 and 3.0 ms, in another embodiment between 0.75 and 2.5 ms, in another embodiment between 0.8 and 2.0 ms, or in another embodiment between 0.9 and 1.75 ms.

Embodiment 19. The method according to any of Embodiment 2 to Embodiment 18, where t_meas2 ranges between 5 and 35% of t_period2, in another embodiment between 10 and 30% of t_period2, in another embodiment between 15 and 25% of t_period2, or in another embodiment between 15 and 22% of t_period2.

Embodiment 20. The method according to any of Embodiment 2 to Embodiment 19, where ITP2 ranges between 12 and 45 A, in another embodiment between 15 and 40 A, in another embodiment between 17 and 35 A, in another embodiment between 20 and 30 A, or in another embodiment between 22 and 27 A.

Embodiment 21. The method according to any of Embodiment 2 to Embodiment 20, where in step C) is
if T_meas≤T_exp then t_heat2=2−80% of (t_period2−t_meas2) and t_wait2=t_period2t_meas2t_heat2, if not t_heat2=0-80% of (t_period2−t_meas2) and t_wait2=t_period2−t_meas2−t_heat2;
in another embodiment if T_meas≤ T_exp then t_heat2=4−60% of (t_period2−t_meas2) and t_wait2=t_period2−t_meas2−t_heat2, if not t_heat2=0-60% of (t_period2−t_meas2) and t_wait2=t_period2−t_meas2−t_heat2;
in another embodiment if T_meas≤ T_exp then t_heat2=6−40% of (t_period2−t_meas2) and t_wait2=t_period2−t_meas2−t_heat2, if not t_heat2=0-40% of (t_period2−t_meas2) and t_wait2=t_period2−t_meas2−t_heat2;
in another embodiment if T_meas≤ T_exp then t_heat2=8−20% of (t_period2−t_meas2) and t_wait2=t_period2−t_meas2−t_heat2, if not t_heat2=0-20% of (t_period2−t_meas2) and t_wait2=t_period2−t_meas2−t_heat2; and
in another embodiment if T_meas≤T_exp then t_heat2=8−15% of (t_period2−t_meas2) and t_wait2=t_period2−t_meas2−t_heat2, if not t_heat2=0% of (t_period2−t_meas2) and t_wait2=t_period2−t_meas2−t_heat2.

Embodiment 22. The method according to any of the previous Embodiments, where the resistance of the drug foil substrate (205) ranges between 1 and 200 mΩ, in another embodiment between 5 and 100 mΩ in another embodiment between 10 and 75 mΩ, in another embodiment between 12 and 50 mΩ, in another embodiment between 12 and 20 mΩ, or in another embodiment between 13 and 20 mΩ.

Embodiment 23. The method according to any of the previous Embodiments, where the drug foil substrate (205) is a metallic drug foil substrate (205).

Embodiment 24. The method according to the previous Embodiment, where the metallic drug foil substrate (205) is a stainless-steel drug foil substrate (205).

Embodiment 25. The method according to any of the previous Embodiments, where the temperature sensor is selected between measurement of electrical resistance across the drug foil substrate (205), optical measurement, and/or direct contact measurement with a thermocouple.

Embodiment 26. The method according to the previous Embodiment, where the temperature sensor is measurement of electrical resistance across the drug foil substrate (205).

Embodiment 27. The method according to the previous Embodiment, where T_meas is measured applying a measurement current I_meas to the drug foil substrate (205) and I_meas ranges between 0.1 and 20 A, in another embodiment between 0.5 and 15 A, in another embodiment between 1 and 10 A, in another embodiment between 1.5 and 7 A, or in another embodiment between 2 and 5 A.

Embodiment 28. The method according to any of Embodiment 26 to Embodiment 27, where the predetermined temperature vs. resistance relationship of the drug foil substrate (205) is stored in a memory (230) of the disposable cartridge (200).

Embodiment 29. The method according to any of the previous Embodiments, where the temperature sensor is measurement of electrical resistance across the drug foil substrate (205) and T_meas is measured applying a measurement current I_meas to the drug foil substrate (205) and I_meas ranges between 0.1 and 20 A, in another embodiment between 0.5 and 15 A, in another embodiment between 1 and 10 A, in another embodiment between 1.5 and 7 A, or in another embodiment between 2 and 5 A.

Embodiment 30. The method according to any of the previous Embodiments, where the HSTP1; tTTP1, and/or HtTP1 are stored in a memory (230) of the disposable cartridge (200).

Embodiment 31. The method according to any of Embodiment 2 to Embodiment 30, where the HSTP2; tTTP2, and/or HtTP2 are stored in a memory (230) of the disposable cartridge (200).

Embodiment 32. The method according to any of the previous Embodiments, where the voltage is from 3 to 13 V, in another embodiment is the voltage is from 3 to 4 V, in another embodiment the voltage is from 6 to 8 V, or in another embodiment the voltage is from 10 to 12 V.

Embodiment 33. A disposable cartridge (200) configured to perform the method of any of the previous Embodiments and suitable to be connected to the handheld controller (100) of any of Embodiment 39 to Embodiment 45 including:
A. an air inlet (220) at one end of the airway (203);
B. an air outlet (202), configured as a mouthpiece, at another end of the airway (203);
C. a drug foil substrate (205) having an impermeable surface, with or without perforations, placed within the airway (203);
D. a drug foil substrate support (204);
E. a solid drug film (207) coated on the drug foil substrate (205); and
F. electrical (302) and/or data connections (301) between the disposable cartridge (200), and the handheld controller (100) of any of Embodiment 39 to Embodiment 45.

Embodiment 34. The disposable cartridge (200) of the previous Embodiment, further including a memory (230) and data connections (301) between the memory and the handheld controller (100) of any of Embodiment 39 to Embodiment 45.

Embodiment 35. The disposable cartridge (200) of the previous Embodiment, where the memory (230) is a long-term memory (including volatile, non-volatile, or semi-volatile memory), a bar code, a QR code or RFID.

Embodiment 36. The disposable cartridge (200) of any of Embodiment 34 to Embodiment 35, where the memory (230) includes the temperature vs. resistance relationship of the drug foil substrate (205).

Embodiment 37. The disposable cartridge (200) of any of Embodiment 33 to Embodiment 36, where the memory (230) includes the HSTP1; tTTP1, and/or HtTP1.

Embodiment 38. The disposable cartridge (200) of any of Embodiment 33 to Embodiment 37, where the memory (230) includes the HSTP2; tTTP2, and/or HtTP2.

Embodiment 39. A handheld controller (100) configured to perform the method of any of Embodiment 1 to Embodiment 31 and suitable to be connected to the disposable cartridge (200) of any of Embodiment 33 to Embodiment 38 including:
A. at least one battery (101)
B. at least one microcontroller (102); and
C. electrical (302) and/or data connections (301) between at least one battery (101), at least one microcontroller (102), and the disposable cartridge (200) of any of Embodiment 33 to Embodiment 38.

Embodiment 40. The handheld controlled of the previous Embodiment where at least one battery (101) is capable of providing between 3 and 12 V, in another embodiment between 4 and 11 V, in another embodiment between 5 and 10 V, in another embodiment between 6 and 10 V, or in another embodiment between 7 and 9 V.

Embodiment 41. The handheld controller (100) of any of Embodiment 39 to Embodiment 40 where at least one battery (101) is capable of delivering peak currents higher than 30 A, in another embodiment higher than 50 A, in another embodiment higher than 75 A, in another embodiment higher than 100 A, in another embodiment higher than 125 A, in another embodiment higher than 150 A, or in another embodiment higher than 175 A.

Embodiment 42. The handheld controller (100) of any of Embodiment 39 to Embodiment 41, which further includes an airflow sensor which triggers the heating of the drug foil substrate (205).

Embodiment 43. The handheld controller (100) of the previous Embodiment, where the airflow sensor is selected from a differential pressure sensor, one or more thermistors, an air flow or sail switch, a hot wire anemometer, or vane anemometer.

Embodiment 44. The handheld controller (100) of any of Embodiment 42 to Embodiment 43, where the airflow sensor triggers the heating of the drug foil substrate at an airflow higher than 7 L/min; in another embodiment higher than 14 L/min; in another embodiment higher than 20 L/min; in another embodiment higher than 30 L/min, or in another embodiment the airflow is between 7 and 35 L/min.

Embodiment 45. A handheld controller (100) of any of Embodiment 39 to Embodiment 44 or a disposable cartridge (200) of any of Embodiment 33 to Embodiment 38 for use in therapy.

Embodiment 46. The method according to any of Embodiment 1 to Embodiment 31, handheld controller (100) of any of Embodiment 39 to Embodiment 45 or the disposable cartridge (200) of any of Embodiment 33 to Embodiment 38, for use in a condition or episode where when the drug in the solid drug film (207) is selected from:
A. loxapine or its pharmaceutically acceptable salts, the condition or episode is agitation, including:
   i. rapidly controlled mild to moderate agitation in adults with schizophrenia or bipolar disorder, or
   ii. acute agitation associated with schizophrenia or bipolar disorder in adults;
B. alprazolam, estazolam or its pharmaceutically acceptable salts, the condition or episode is epilepsy, where epilepsy includes seizures;
C. fentanyl or its pharmaceutically acceptable salts, the condition or episode is breakthrough pain;
D. zaleplon, almorexant or its pharmaceutically acceptable salts, the condition or episode is a sleep disorder including:
   i. middle of the night awakening, or
   ii. middle of the night insomnia;
E. apomorphine, pergolide, ropinirole, pramipexol, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
   i. nausea,
   ii. vomiting or
   iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexol, or rotigotine the condition or episode is restless legs syndrome.

Embodiment 47. A method of treatment for a condition or episode which includes administering a drug included in the solid drug film (207) coated on at least a portion of the drug foil substrate (205) of the method according to any of Embodiment 1 to Embodiment 31, handheld controller (100) of any of Embodiment 39 to Embodiment 45 or the disposable cartridge (200) of any of Embodiment 33 to Embodiment 38, where when the drug in the solid drug film (207) is selected from:
A. loxapine or its pharmaceutically acceptable salts, the condition or episode is agitation, including:
   i. rapidly controlled mild to moderate agitation in adults with schizophrenia or bipolar disorder, or
   ii. acute agitation associated with schizophrenia or bipolar disorder in adults;
B. alprazolam, estazolam or its pharmaceutically acceptable salts, the condition or episode is epilepsy, where epilepsy includes seizures;
C. fentanyl or its pharmaceutically acceptable salts, the condition or episode is breakthrough pain;
D. zaleplon, almorexant or its pharmaceutically acceptable salts, the condition or episode is a sleep disorder including:
   i. middle of the night awakening, or
   ii. middle of the night insomnia;
E. apomorphine, pergolide, ropinirole, pramipexol, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
   i. nausea,
   ii. vomiting or
   iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexol, or rotigotine the condition or episode is restless legs syndrome.

Embodiment 48. Use of the method according to any of Embodiment 1 to Embodiment 31, previous embodiments, handheld controller (100) of any of Embodiment 39 to Embodiment 45 (previous embodiments) or the disposable cartridge (200) of any of Embodiment 33 to Embodiment 38 for the manufacturing of a medicament for the treatment of a condition or episode; where when the drug in the solid drug film (207) is selected from:
A. loxapine or its pharmaceutically acceptable salts, the condition or episode is agitation, including:
   i. rapidly controlled mild to moderate agitation in adults with schizophrenia or bipolar disorder, or
   ii. acute agitation associated with schizophrenia or bipolar disorder in adults;
B. alprazolam, estazolam or its pharmaceutically acceptable salts, the condition or episode is epilepsy, where epilepsy includes seizures;
C. fentanyl or its pharmaceutically acceptable salts, the condition or episode is breakthrough pain;
D. zaleplon, almorexant or its pharmaceutically acceptable salts, the condition or episode is a sleep disorder including:
   i. middle of the night awakening, or
   ii. middle of the night insomnia;
E. apomorphine, pergolide, ropinirole, pramipexol, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
   i. nausea,
   ii. vomiting or
   iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexol, or rotigotine, the condition or episode is restless legs syndrome.

Embodiment 49. A medicament including a drug selected from: loxapine, alprazolam, estazolam, fentanyl, zaleplon, almorexant, apomorphine, pergolide, ropinirole, pramipexol, granisetron, ondansetron, palonosetron, nicotine, nicotine meta-salicylate, rotigotine, or its pharmaceutically acceptable salts for use in the method according to any of Embodiment 1 to Embodiment 31, (previous embodiments) handheld controller (100) of any of Embodiment 39 to Embodiment 45 or the disposable cartridge (200) of any of Embodiment 33 to Embodiment 38.

Embodiment 50. A drug deposited on a drug foil substrate (205) of the method according to any of Embodiment 1 to Embodiment 31, handheld controller (100) of any of Embodiment 39 to Embodiment 45 or the disposable cartridge (200) of any of Embodiment 33 to Embodiment 38 for use in a condition or episode, when the drug is:
A. loxapine or its pharmaceutically acceptable salts, the condition or episode is agitation, including:
   i. rapidly controlled mild to moderate agitation in adults with schizophrenia or bipolar disorder, or
   ii. acute agitation associated with schizophrenia or bipolar disorder in adults;
B. alprazolam, estazolam or its pharmaceutically acceptable salts, the condition or episode is epilepsy, where epilepsy includes seizures;
C. fentanyl or its pharmaceutically acceptable salts, the condition or episode is breakthrough pain;
D. zaleplon, almorexant or its pharmaceutically acceptable salts, the condition or episode is a sleep disorder including:
   i. middle of the night awakening, or
   ii. middle of the night insomnia;
E. apomorphine, pergolide, ropinirole, pramipexol, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
   i. nausea,
   ii. vomiting or
   iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexol, or rotigotine, the condition or episode is restless legs syndrome.

### EXAMPLES

### IR Temperature Measurement

The temperature is measured using FLIR Systems infrared cameras, Thermacam SC3000 and A655sc. The SC3000 infrared camera uses quantum well infrared photodetector technology, and the A655sc uses an uncooled microbolometer detector. These cameras are adequate for high sensitivity and accuracy and captures images up to 180 and 200 Hz, respectively. Temperature is calculated based on the amount of emitted infrared light. The camera was calibrated by heating the metal cylinders (resistive heating with a constant current DC power supply) to various steady-state temperatures between 20° and 400° C. and measuring the actual temperature with calibrated thermocouples (Omega, Stamford, CT).

### Device 1: SS, Electrical Bench-Top Screening Device

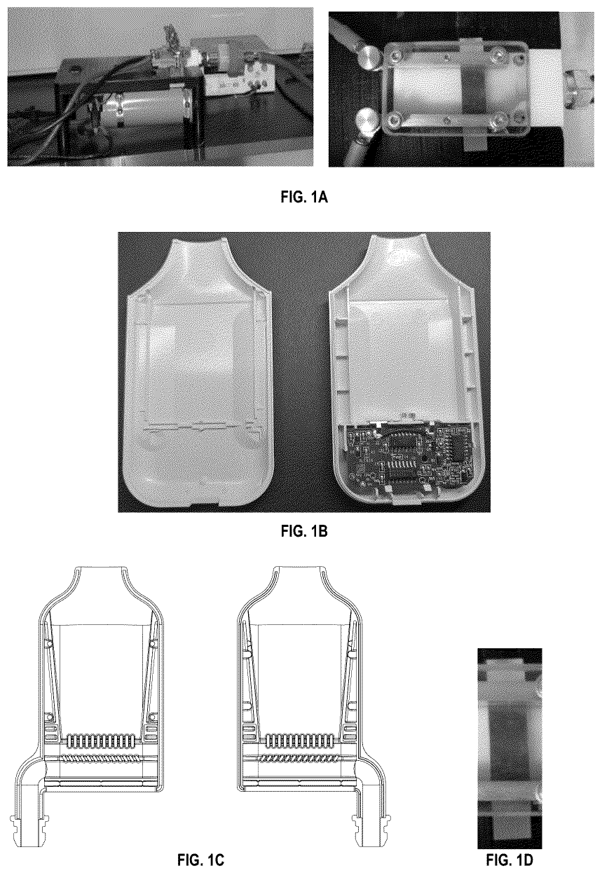

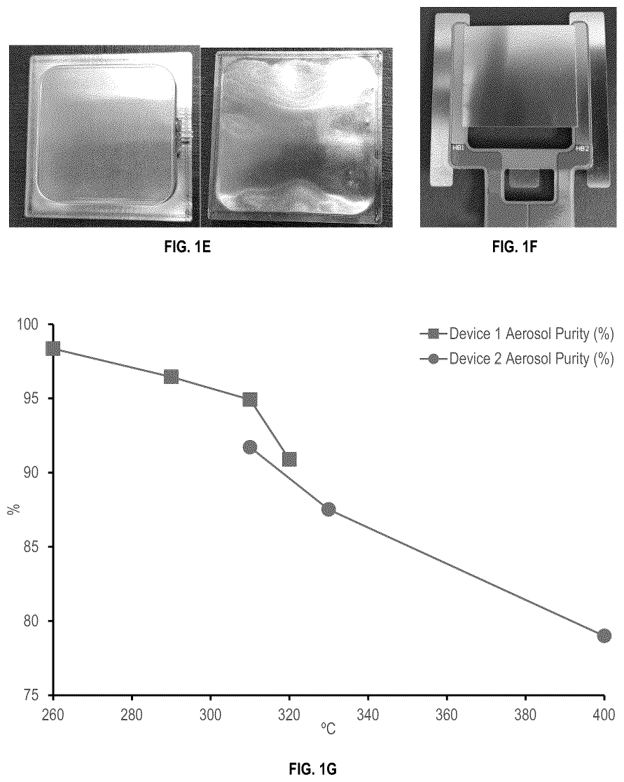

A testing device where a Heat Source 1 is mechanically and electrically attached as shown in FIG. 1A. Device 1 has an air inlet and air outlet and a 1 F capacitor (Phoenix Gold Titanium series) to be discharged to resistively heat the substrate (<0.2 sec) and facilitate rapid aerosol formation with a cross-foil airflow of ˜ 30 L/min. Calibration for temperature versus applied voltage was determined with a thermocouple spot-welded to the foil surface.

### Device 2: Handheld Housing 1

A 100 mm long device such as that in FIG. 1B, the same shape as that of the Adasuve® device, moulded from PermaStat® resin (antistatic material).

### Device 3: Handheld Housing 2

An 89 mm long device such as that in FIG. 1C moulded from PermaStat® resin (antistatic material).

### Heat Source 1: Testing Electric Heating

A 12.7×63.5, 0.127 mm 304-stainless steel drug foil substrate attached to Device 1 with connexions to receive electrical power from an electrical source. Devices were actuated with a constant airflow of ˜ 30 L/min. An example is depicted in FIG. 1D.

### Heat Source 2: Chemical Heat Package

A 47.2×47.2×0.38 mm 304-stainless steel drug foil substrate is attached to a chemical heat package connected to Device 2. One chemical heat package is shown in FIG. 1E.

Information on chemical heating packs can be found in WO2004104492, incorporated herein by reference.

In chemical heat package devices, an exothermic thermite reaction from the reactants inside of the heat package rapidly propagated (˜ 0.1 s) and generated heat nearly instantaneously, which vaporized the drug film. The reactants are calibrated to heat the drug foil substrate to a given temperature. Upper and lower housings were welded prior to vaporization. Devices were actuated with a constant airflow of ˜ 30 L/min.

### Heat Source 3: Electric Heating

A 31×28×0.127 mm 304-stainless steel drug foil substrate with electrical connections, such as the one in FIG. 1F, is connected to Device 3. Device 3 is attached to a handheld controller such as the one described in WO2019152873.

### Comparative Example 1

mg apomorphine HCl (APO) was coated on 2.27 cm² (0.44 mg/cm²) on the drug foil substrate of Heat Source 1 or 2 and heated to the temperatures specified in the table below.

| Heat | Substrate | | Aerosol Purity | |
|---|---|---|---|---|
| Source | Temp. (° C.) | % Vaporization | (%) | n |
| 1 | 260 | 40.57 ± 9.34 | 98.35 ± 0.64 | 2 |
| 1 | 290 | 58.03 ± 12.15 | 96.45 ± 0.93 | 2 |
| 1 | 310 | 59.69 ± 0.34 | 94.92 ± 1.12 | 4 |
| 1 | 320 | 66.14 ± 0.95 | 90.89 ± 0.78 | 2 |
| 2 | 310 | 48.32 ± 16.77 | 91.71 ± 1.62 | 7 |
| 2 | 330 | 61.58 ± 2.07 | 87.51 ± 0.18 | 3 |
| 2 | 400 | 49.07 ± 1.12 | 78.9 1.95 | 3 |

This data is plotted in FIG. 1G, which generally shows that the higher the temperature the higher the % vaporization and the lower the aerosol purity.

### Example 1

An uncoated drug foil substrate of Heat Source 3 but modified to incorporate a window of calcium fluoride (which is transparent to IR light) was heated using the method where if T_meas≤ T_exp then t_heat1=t_period1−t_meas1 else t_wait1=t_period1−t_meas1. In this example HSTP1 is 1.5° C./ms, tTTP1 is 350° C., t_period_1 is 1.3 ms and t_meas1 is 0.3 ms. The temperature is measured using a resistance of the drug foil substrate vs. temperature relationship using a circuit as that in FIG. 3B with an error of ±3° C.

When T_meas is close to tTTP1 then when T_meas≤T_exp then t_heat1=10% (t_period1−t_meas1) else t_wait1=t_period1−t_meas1 in order to have a smoother curve and prevent overheating the drug foil substrate. That is why the plot in FIG. 10 appears as a soft curve before starting TP2.

For TP2, HSTP2 is −0.05° C./ms, tTTP2 is 313° C.

FIG. 10 shows a plot of T_exp (dashed line) and the temperature measured as described in IR temperature measurement. Measured temperature closely follows T_exp, the difference is due different measurement methods.

### Example 2

The same device and conditions as in Example 1 were used but with HSTP1=2.0° C./ms, tTTP1=309° C., HSTP2=0° C./ms, and tTTP2 is 309° C. nine different uncoated drug foil substrates were used.

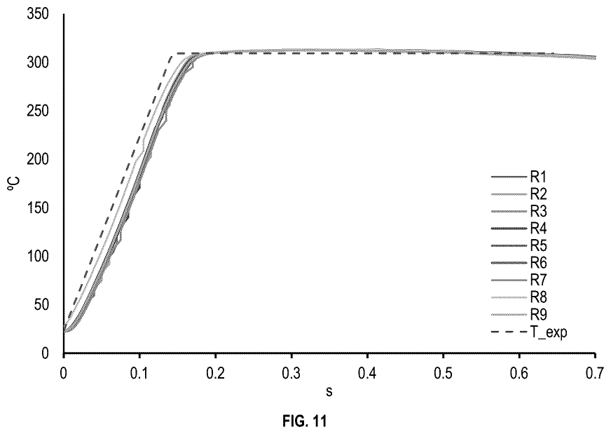

FIG. 11 shows a plot of T_exp (dashed line) and the temperature measured as described in IR temperature measurement. Measured temperature closely follows T_exp, the difference is due different measurement methods.

The reference numbers in the figures mean:
100: Handheld controller.
101: Batteries.
102: Microcontroller.
103: Air inlet controller extension.
200: Single dose disposable cartridge.
202: Air outlet adapted as a mouthpiece.
203: Airway defined by the internal walls.
204: Drug foil substrate support.
205: Drug foil substrate.
207: Solid drug film.
220: Air inlet.
230: Disposable cartridge memory.
301: Data connections between the handheld controller (100) and disposable cartridge (200).
302: Electric connections between the handheld controller (100) and disposable cartridge (200).

### LIST OF VARIABLES

T_meas: Measured temperature during any t_meas1 or t_meas2.
T_exp: Expected temperature at any given moment when following TP1 or TP2.
TP1: First temperature vs. time profile.
HSTP1: Average heating slope in TP1.
tTTP1: Target temperature in TP1.
HtTP1: Heating time in TP1.
t_period1: Length of the feedback periods in which is divided TP1.
t_heat1: Time while the drug foil substrate (205) is heated when following TP1, it is a fraction of t_period1.
t_wait1: Time while the drug foil substrate (205) is not heated neither its temperature is measured when following TP1, it is a fraction of t_period1.
t_meas1: Time while the temperature of drug foil substrate (205) is measured when following
TP1, it is a fraction of t_period1.
ITP1: Electric current used to heat the drug foil substrate (205) when following TP1.
TP2: Second temperature vs. time profile.
HSTP2: Heating slope in TP2.
tTTP2: Target temperature in TP2.
HtTP2: Heating time in TP2.
t_period2: Length of the feedback periods in which is divided TP2.
t_heat2: Time while the drug foil substrate (205) is heated when following TP2, it is a fraction of t_period2.
t_wait2: Time while the drug foil substrate (205) is not heated neither its temperature is measured when following TP2, it is a fraction of t_period2.
t_meas2: Time while the temperature of drug foil substrate (205) is measured when following
TP2, it is a fraction of t_period2.
ITP2: Electric current used to heat the drug foil substrate (205) when following TP2.
I_meas: Electric current used to measure the temperature of the drug foil substrate (205) when using the measurement of electrical resistance across the drug foil substrate (205) technique.

Various embodiments are called out for example purposes and are in no way meant to be limiting.

1. A method of electrically heating a drug foil substrate coated with a solid drug film, that is placed in a disposable cartridge following a first temperature versus time profile (TP1) to generate a condensation aerosol;
the disposable cartridge is configured to be connected to a handheld controller;
TP1 comprises:
a heating function with an average slope (HSTP1) that ranges between 1 and 5° C./ms; and
a target temperature ((TTP1) that ranges between 20° and 550° C., and/or
a heating time (HtTP1) that ranges between 40 and 550 ms;
the method comprising:
a. a regulation phase comprising:
applying an electric current (ITP1) to the drug foil substrate during t_heat1 and not applying any current during t_wait1,
not applying any current during t_wait1 and applying an electric current (ITP1) to the drug foil substrate during t_heat1, or
the previous t_wait1 and t_heat1 are distributed in alternate sections;
b. determining the temperature of the drug foil substrate (T_meas) during t_meas1 using a temperature sensor;
c. when T_meas≤ T_exp then
t_heat1=1-100% of (t_period1−t_meas1) and t_wait1=t_period1−t_meas1-t_heat1,
when not
t_heat1=0-100% of (t_period1−t_meas1) and t_wait1=t_period1−t_meas1-t_heat1,
where T_exp is based on TP1; and
d. repeating steps a) to d) to match TP1 until tTTP1 is reached and/or HtTP1 has elapsed;
wherein:
the control period time (t_period1) ranges between 0.5 and 5 ms,
the measurement time t_meas1 ranges between 1-40% of t_period1,
T_exp is the temperature expected at any given time point based on TP1,
t_period1=t_heat1+t_wait1+t_meas1,
t_period1 is the length of time of each cycle of measuring temperature and optionally heating the drug foil substrate,
t_heat1 is the time the drug foil substrate is heated within each t_period1,
t_wait1 is the time the drug foil substrate is not heated within each t_period1,
t_meas1 is the time the temperature of the drug foil substrate is measured within each t_period1, and
the electric current (ITP1) is between 30 and 400 A.

2. The method of claim 1, further comprising a second temperature vs. time profile (TP2) after the end of TP1;
wherein TP2 comprises:
a heating function with an average slope (HSTP2) that ranges between −0.15 and 0.15° C./ms; and
a target temperature ((TTP2) that ranges between 20° and 550° C., and/or
a heating time (HtTP2) that ranges between 0.5 and 2000 ms;
the method further comprising:
A. a regulation phase comprising:
applying an electric current (ITP2) to the drug foil substrate during t_heat2 and not applying any current during t_wait2,
not applying any current during t_wait2 and applying an electric current (ITP2) to the drug foil substrate during t_heat2, or
the previous t_wait2 and t_heat2 are distributed in alternate sections;
B. determining the temperature drug foil substrate (T_meas) during t_meas2 using a temperature sensor;
C. when T_meas≤ T_exp then
t_heat2=1-100% of (t_period2−t_meas2) and t_wait2=t_period2−t_meas2−t_heat2,
when not
t_heat2=0-100% of (t_period2−t_meas2) and t_wait2=t_period2−t_meas2−t_heat2,
wherein T_exp is based on TP2; and
D. repeating steps A) to D) to match TP2 until (TTP2 is reached and/or HtTP2 has elapsed;
wherein
the control period time t_period2 ranges between 0.5 and 5 ms,
the measurement time t_meas2 ranges between 1-40% of t_period2,
t_period2=t_heat2+t_wait2+t_meas2,
t_period2 is the length of time of each cycle of measuring temperature and optionally heating the drug foil substrate,
t_heat2 is the time the drug foil substrate is heated within each t_period2,
t_wait2 is the time the drug foil substrate is not heated within each t_period2,
t_meas2 is the time the temperature of the drug foil substrate is measured within each t_period2, and
the electric current ITP2 ranges between 10 and 50 A.

3. The method according to claim 1, wherein t_period1 and/or t_period2 range between 0.55 and 4.0 ms.

4. The method according to claim 1, wherein t_meas1 and/or t_meas2 range between 5 and 35% of t_period1.

5. The method according to claim 1, wherein ITP1 ranges between 50 and 290 A.

6. The method according to claim 1, wherein when T_meas is higher than tTTP1−50° C. and lower than (TTP1-20° C., then c) is if T_meas≤T_exp then t_heat1=70-90% of (t_period1−t_meas1) and t_wait1=t_period1−t_meas1−t_heat1, if not t_heat1=0% of (t_period1−t_meas1) and t_wait1=t_period1−t_meas1−t_heat1;

7. The method according to claim 1, wherein when T_meas is higher than tTTP1−20° C., then c) is if T_meas≤ T_exp then t_heat1=40-60% of (t_period1−t_meas1) and t_wait1=t_period1−t_meas1−t_heat1, if not t_heat1=0% of (t_period1−t_meas1) and t_wait1=t_period1−t_meas1−t_heat1.

8. The method according to claim 1, when T_meas is lower than tTTP1−50° C., step c) is if T_meas≤ T_exp then t_heat1=2-100% of (t_period1−t_meas1) and t_wait1=t_period1−t_meas1−t_heat1, if not t_heat1=0-80% of (t_period1−t_meas1) and t_wait1=t_period1−t_meas1−t_heat1.

9. The method according to claim 2, wherein ITP2 ranges between 12 and 45 A.

10. The method according to claim 2, wherein in step C) is if T_meas≤ T_exp then t_heat2=2-80% of (t_period2−t_meas2) and t_wait2=t_period2−t_meas2−t_heat2, if not t_heat2=0-80% of (t_period2−t_meas2) and t_wait2=t_period2−t_meas2−t_heat2.

11. The method according to claim 1, wherein the temperature sensor uses a measurement of electrical resistance across the drug foil substrate, and T_meas is measured by applying a measurement current I_meas to the drug foil substrate and I_meas ranges between 0.1 and 20 A.

12. The method according to claim 11, wherein the predetermined temperature vs. resistance relationship of the drug foil substrate is stored in a memory of the disposable cartridge.

13. A disposable cartridge configured to perform the method of claim 1, and configured to be connected to a handheld controller, the disposable cartridge comprising:
an air inlet at one end of the airway;
an air outlet, configured as a mouthpiece, at another end of the airway;
a drug foil substrate having an impermeable surface, with or without perforations, placed within the airway;
a drug foil substrate support;
a solid drug film coated on the drug foil substrate; and
electrical and/or data connections between the disposable cartridge and the handheld controller, wherein the handheld controller comprises at least one battery;
at least one microcontroller; and
electrical and/or data connections between the at least one battery, the at least one microcontroller, and the disposable cartridge.

14. A handheld controller configured to perform the method claim 1 and configured to be connected to a disposable cartridge comprising:
at least one battery;
at least one microcontroller; and
electrical and/or data connections between the at least one battery, the at least one microcontroller, and the disposable cartridge, wherein the disposable cartridge comprises: an air inlet at one end of the airway; an air outlet, configured as a mouthpiece, at another end of the airway; a drug foil substrate having an impermeable surface, with or without perforations, placed within the airway; a drug foil substrate support; a solid drug film coated on the drug foil substrate; and electrical and/or data connections between the disposable cartridge and the handheld controller, wherein the handheld controller comprises at least one battery; at least one microcontroller; and electrical and/or data connections between the at least one battery, the at least one microcontroller, and the disposable cartridge.

15. The method according to claim 1, for use in a condition or episode wherein when the drug in the solid drug film is selected from the group consisting of:
A. loxapine or its pharmaceutically acceptable salts, the condition or episode is agitation, comprising:
rapidly controlled mild to moderate agitation in adults with schizophrenia or bipolar disorder, or
acute agitation associated with schizophrenia or bipolar disorder in adults;
B. alprazolam, estazolam or its pharmaceutically acceptable salts, the condition or episode is epilepsy, wherein epilepsy comprises seizures;
C. fentanyl or its pharmaceutically acceptable salts, the condition or episode is breakthrough pain;
D. zaleplon, almorexant or its pharmaceutically acceptable salts, the condition or episode is a sleep disorder comprising:
i. middle of the night awakening, or
ii. middle of the night insomnia;
E. apomorphine, pergolide, ropinirole, pramipexol, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
i. nausea,
ii. vomiting or
iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexol, or rotigotine, the condition or episode is restless legs syndrome.

16. A medicament comprising a drug selected from the group consisting of: loxapine, alprazolam, estazolam, fentanyl, zaleplon, almorexant, apomorphine, pergolide, pramipexole, ropinirole, pramipexol, granisetron, ondansetron, palonosetron, nicotine, nicotine meta-salicylate, rotigotine, or its pharmaceutically acceptable salts for use in the method according to claim 1.

17. A drug deposited on a drug foil substrate of the method according to claim 1, for use in a condition or episode, wherein when the drug is selected from the group consisting of:
A. loxapine or its pharmaceutically acceptable salts, the condition or episode is agitation, comprising:
i. rapidly controlled mild to moderate agitation in adults with schizophrenia or bipolar disorder, or
ii. acute agitation associated with schizophrenia or bipolar disorder in adults;
B. alprazolam, estazolam or its pharmaceutically acceptable salts, the condition or episode is epilepsy, wherein epilepsy comprises seizures;
C. fentanyl or its pharmaceutically acceptable salts, the condition or episode is breakthrough pain;
D. zaleplon, almorexant or its pharmaceutically acceptable salts, the condition or episode is a sleep disorder comprising:
i. middle of the night awakening, or
ii. middle of the night insomnia;
E. apomorphine, pergolide, ropinirole, pramipexol, or its pharmaceutically acceptable salts, the condition or episode is Parkinson's disease, off-episodes in Parkinson's disease, and/or idiopathic Parkinson's disease;
F. granisetron, ondansetron, palonosetron or its pharmaceutically acceptable salts, the condition or episode is:
i. nausea,
ii. vomiting or
iii. cyclic vomiting syndrome;
G. nicotine or its pharmaceutically acceptable salts including nicotine meta-salicylate, the condition or episode is nicotine craving and/or effecting cessation of smoking; or
H. ropinirole, pramipexol, or rotigotine, the condition or episode is restless legs syndrome.
